(19) United States
(12) Patent Application Publication
(10) Pub. No.: US 2026/0034076 A1
(43) Pub. Date: Feb. 5, 2026

(54) COMPOSITIONS AND METHODS FOR TREATING PANCREATIC DISEASE INCLUDING TYPE 3C DIABETES

(71) Applicant: Jason Patrick McDevitt, Williamsburg, VA (US)

(72) Inventor: Jason Patrick McDevitt, Williamsburg, VA (US)

(21) Appl. No.: 19/278,708

(22) Filed: Jul. 23, 2025

Related U.S. Application Data
(60) Provisional application No. 63/677,529, filed on Jul. 31, 2024.

(51) Int. Cl. A61K 31/155; A61K 31/197

(52) U.S. Cl. CPC A61K 31/155; A61K 31/197

(57) ABSTRACT

Methods and compositions are described for treating pancreatic disease, including chronic pancreatitis and/or type 3c diabetes. A pharmaceutical composition of proglumide and metformin is administered to a human subject at a dose of between 250 mg and 2,400 mg proglumide per day, and between 500 mg and 2,500 mg metformin per day.

### CROSS-REFERENCE TO RELATED APPLICATIONS

The present application claims priority under 35 U.S.C. § 119 (e) to (i) U.S. Provisional Patent Application No. 63/677,529, filed Jul. 31, 2024. The entire disclosure of this application is incorporated by reference herein.

### BACKGROUND OF THE INVENTION

Proglumide is a drug which has been approved for use in many countries as a treatment for stomach ulcers. It is not approved for use in the United States, and is no longer frequently prescribed internationally, with newer anti-ulcer drugs preferred. Proglumide inhibits gastrointestinal mobility and acts as a cholecystokinin antagonist, blocking both the CCK_{A} and CCK_{B} subtypes.

Cholecystokinin (CCK) is widely distributed in the central nervous system, and its levels increase with chronic opiate administration. Increased levels of CCK are believed to reduce the analgesic effect of opiates and also are believed to be at least partially responsible for tolerance to opiate analgesics such as morphine.

Since proglumide is a CCK antagonist (albeit a moderate, non-selective one), it has been widely studied for its potential role in enhancing opiate efficacy and/or reducing opiate tolerance. For example, studies have determined that proglumide enhances the analgesic efficacy of morphine in treating human patients with chronic pain and that proglumide potentiates morphine analgesia in both opiate-naïve and morphine-tolerant rats. Other studies have observed no difference between treatment and control groups in studies examining whether proglumide augments morphine analgesia in cancer pain management and in post-operative patients.

A recent human pharmacokinetic study (Pharmaceutics (2022), 14, p. 627) conducted by Hsu et al. determined that peak serum proglumide levels were reached in about an hour after subjects were administered 400 mg proglumide capsules. Peak urine concentration was reached in three hours, and serum proglumide levels had fallen to nearly baseline levels at 24 hours.

Pancreatitis is a condition characterized by inflammation of the pancreas. There are two types of pancreatitis: acute pancreatitis and chronic pancreatitis. Acute pancreatitis can resolve quickly, although it can also become chronic pancreatitis.

CCK signaling has been thought to play a role in both acute pancreatitis and chronic pancreatitis. As a result, CCK antagonists have been widely studied in both humans and animals as potential treatments for pancreatitis.

In a double-blind study for treatment of acute pancreatitis, 189 patients were treated with three different doses of the selective CCK1 antagonist loxiglumide, administered intravenously twice daily (Ochi K, Harada H, Satake K. “Clinical evaluation of cholecystokinin-A-receptor antagonist (loxiglumide) for the treatment of acute pancreatitis. A preliminary clinical trial. Study Group of Loxiglumide in Japan”. Digestion 1999; 60:81-85). There were statistically insignificant differences between the groups for the measures of pain disappearance, nausea, vomiting, and changes in serum amylase levels, although serum lipase levels returned to normal levels more quickly in the high-dose group. However, the study lacked a placebo control necessary to determine efficacy.

In a double-blind, placebo-controlled study for treatment of chronic pancreatitis, 207 patients were treated with three different doses (300, 600, or 1,200 mg/day) of the selective CCK1 antagonist loxiglumide (Shiratori K, Takeuchi T, Satake K, Matsuno S. “Clinical evaluation of oral administration of a cholecystokinin-A receptor antagonist (loxiglumide) to patients with acute, painful attacks of chronic pancreatitis: a multicenter dose-response study in Japan”. Pancreas 2002; 25: e1-e5. [PubMed: 12131781]). In the 600 mg group, back/abdominal pain, serum amylase and trypsin levels decreased significantly. In all three groups, abdominal tenderness improved.

In mouse models of pancreatitis, Nadella et al. (Dig Dis Sci. 2020 May; 65 (5): 1376-1384) found that the non-selective CCK inhibitor proglumide reduced fibrosis and inflammation. The mice received proglumide in their water for 18 weeks.

In 2000, McCleane observed an analgesic effect with proglumide in chronic pancreatitis (McCleane, Gary J., “The Cholecystokinin Antagonist Proglumide Has an Analgesic Effect in Chronic Pancreatitis”, Pancreas 21 (3): p 324-325, October 2000).

In a recent human clinical trial, Ciofoaia et al. found that proglumide reduced pain and biomarkers associated with inflammation relative to placebo in subjects with chronic pancreatitis (Ciofoaia V, Chen W, Tarek B W, Gay M, Shivapurkar N, Smith J P. The Role of a Cholecystokinin Receptor Antagonist in the Management of Chronic Pancreatitis: A Phase 1 Trial. Pharmaceutics. 2024; 16 (5): 611).

In a rat model of pancreatitis, Tarpila et al. (Tarpila, E., et al. “Proglumide treatment in bile-induced acute experimental pancreatitis”, Int J Pancreatol 3, 195-202 (1988)) found biochemical changes consistent with a beneficial therapeutic effect by proglumide, but found that survival rate was not improved when using a dose of 100 mg/kg every 8 h for 48 h and was reduced at a dose of 500 mg/kg every 8 h for 48 h. The authors concluded that “the results do not support proglumide as the sole treatment in acute pancreatitis”.

Barrett et al. (British Journal of Pharmacology (2008) 153, p. 1650-1658) evaluated two selective CCK1 receptor antagonists in normal rats and rats with bile duct ligation. They concluded that elevation of plasma amylase and lipase activity in the early stages of obstruction-induced pancreatitis was largely driven by elevation of plasma CCK concentration and activation of CCK1 receptors, suggesting that CCK is a factor in acute pancreatitis in rats.

Taken cumulatively, these human and animal studies are inconclusive but suggest that CCK receptor antagonism, including treatment with the CCK antagonist proglumide, can potentially be effective in treating or preventing pancreatitis.

Chronic pancreatitis often leads to type 3c diabetes, also known as pancreatogenic diabetes. Type 3c diabetes is characterized by exocrine pancreatic insufficiency. Other causes of type 3c diabetes include, for example, pancreatic cancer, pancreatic disease, and cystic fibrosis. Type 3c diabetes is often misdiagnosed as type 2 diabetes.

Metformin has often been used to treat type 3c diabetes, including in patients with type 3c diabetes who have had chronic pancreatitis. In a nationwide cohort study in Denmark, metformin was observed to reduce all-cause mortality and hypoglycemia risk in people with post-pancreatis diabetes mellitus (Davidsen et al., “Metformin treatment is associated with reduced risk of hypoglycaemia, major adverse cardiovascular events, and all-cause mortality in patients with post-pancreatitis diabetes mellitus: a nationwide cohort study”, European Journal of Endocrinology, Volume 190, Issue 1, January 2024, Pages 44-53).

Unfortunately, metformin treatment can itself induce pancreatitis, see for example Alsubaie et al. (Alsubaie, S., & Almalki, M. H. (2013), “Metformin induced acute pancreatitis”, Dermato-Endocrinology, 5 (2), 317-318) and Damughatla et al. (Damughatla A R, Surapaneni S, Wadehra A, et al. (2022), “A Novel Case of Metformin-Induced Pancreatitis in an Individual With Normal Dosing and No Underlying Chronic Kidney Disease”, Cureus 14 (5): e25116). Moreover, metformin can also exacerbate chronic pancreatis (Wadsworth T G, Kiester J, Thompson E G. (2012), “Metformin-Associated Exacerbation of Chronic Pancreatitis: A Possible Adverse Drug-Disease Interaction”, Journal of Pharmacy Technology, 28 (5): 208-210).

While proglumide may be effective in treating chronic pancreatitis, its mechanism of action, as well as experimental results, suggest that proglumide could be counterproductive for treating diabetes. Proglumide is a CCK antagonist. Like the well-known gut hormone GLP-1, CCK promotes satiety and reduces food intake. By blocking the CCK satiety signals, proglumide could potentially lead to increased food consumption, which may affect blood glucose levels. CCK is also known to stimulate insulin secretion. By antagonizing CCK, proglumide might reduce this stimulatory effect, potentially causing higher post-prandial blood glucose levels, particularly in subjects with exocrine pancreatic insufficiency, which is a characteristic of chronic pancreatitis. CCK appears to have a role in post-prandial insulin sensitivity, so CCK antagonists can be expected to reduce insulin sensitivity. Finally, CCK plays a role in slowing gastric emptying. Proglumide's antagonistic effect might lead to faster gastric emptying, which can influence postprandial blood glucose levels.

In a mouse study, Parmar et al. found that proglumide exacerbated hyperglycemia and decreased plasma insulin levels in diabetic mice (N S Parmar, M Tariq, A M Ageel, “Proglumide, a cholecystokinin receptor antagonist, exacerbates alloxan-induced diabetes mellitus in Swiss mice”, Journal of Pharmacy and Pharmacology, Volume 39, Issue 12, December 1987, Pages 1028-1030)

Accordingly, while proglumide is known as a potential treatment for chronic pancreatitis, and chronic pancreatitis often leads to diabetes, proglumide by itself might exacerbate diabetes. Metformin is well-known as a treatment for diabetes, but by itself can exacerbate chronic pancreatitis. There are no drugs currently approved by FDA for either condition. While not all patients with chronic pancreatitis have type 3c diabetes, and while not all patients with type 3c diabetes have chronic pancreatitis, there is significant overlap. A pharmaceutical treatment that improves only one of these conditions, while being neutral or exacerbating the other, is not a good long-term solution.

It would be preferable to develop a pharmaceutical treatment that is effective for both type 3c diabetes and chronic pancreatitis. It would also be preferable to develop a pharmaceutical treatment for type 3c diabetes that can protect against developing pancreatitis. It would also be preferable to develop a pharmaceutical treatment for chronic pancreatitis which reduces the risk of developing diabetes in a patient who does not yet have type 3c diabetes. It would be preferable to develop a pharmaceutical treatment for pancreatic disease such as type 3c diabetes and chronic pancreatitis which also reduces the risk of developing pancreatic cancer.

### SUMMARY OF THE INVENTION

Methods are described for treating a pancreatic disease comprising administering (i) proglumide, or a pharmaceutically acceptable salt thereof, and (ii) metformin, or a pharmaceutically acceptable salt thereof, to a human subject with type 3c diabetes. Methods are described for treating type 3c diabetes comprising administering (i) proglumide, or a pharmaceutically acceptable salt thereof, and (ii) metformin, or a pharmaceutically acceptable salt thereof, to a human subject with type 3c diabetes. Methods are also described for treating chronic pancreatitis comprising administering (i) proglumide, or a pharmaceutically acceptable salt thereof, and (ii) metformin, or a pharmaceutically acceptable salt thereof, to a human subject with chronic pancreatitis. Methods are described for treating a human subject afflicted with both type 3c diabetes and chronic pancreatitis, or with post-pancreatitis diabetes mellitus, comprising administering (i) proglumide, or a pharmaceutically acceptable salt thereof, and (ii) metformin, or a pharmaceutically acceptable salt thereof, to a human subject with both type 3c diabetes and chronic pancreatitis, or with post-pancreatitis diabetes mellitus. According to these methods, the daily dosages of proglumide, or pharmaceutically acceptable salts thereof, are between 250 mg and 2,400 mg, and the daily dosages of metformin, or pharmaceutically acceptable salts thereof, are between 500 mg and 2,500 mg. Dosages can be administered once daily, or multiple times per day. In other embodiments, dosages can be administered on a pulsed basis such as two days on followed by five days off. Extended-release formulations can be utilized.

Pharmaceutical compositions are described which are useful for treating pancreatic disorders, including chronic pancreatitis and/or type 3c diabetes. These pharmaceutical compositions comprise metformin and proglumide, or pharmaceutically acceptable salts thereof. The pharmaceutical compositions comprise between 100 mg and 2,400 mg proglumide, or pharmaceutically acceptable salts thereof, and between 250 mg and 2,500 mg metformin, or pharmaceutically acceptable salts thereof.

Benefits of treatment of pancreatic disease with a combination of metformin and proglumide can include, but are not limited to: reduced risk of developing diabetes, reduced risk of developing pancreatic cancer, reduced risk of exacerbating pancreatitis, improved management of blood sugar levels, reduced hemoglobin A1C percentages, reduced average blood sugar levels, fewer hyperglycemic episodes, fewer hypoglycemic episodes, reduced pain (e.g., reduced pain associated with chronic pancreatitis), reduced pancreatic inflammation, and reduced pancreatic fibrosis.

### DETAILED DESCRIPTION OF THE INVENTION

As used herein, each of the following terms has the meaning associated with it as described below.

Pancreatic diseases, including type 3c diabetes and chronic pancreatitis, present significant challenges in medical treatment. Type 3c diabetes, also known as pancreatogenic diabetes, arises from damage to the pancreas, often due to chronic pancreatitis, pancreatic cancer, or other pancreatic disorders. Chronic pancreatitis, characterized by long-lasting inflammation of the pancreas, can lead to permanent damage, scarring, and exocrine pancreatic insufficiency. These conditions frequently co-occur, complicating treatment strategies and patient outcomes.

Current treatments for type 3c diabetes and chronic pancreatitis have notable limitations. Metformin, a widely used treatment for type 3c diabetes, can induce or exacerbate pancreatitis, posing a risk to patients with or susceptible to pancreatic inflammation. Proglumide, a cholecystokinin (CCK) antagonist, has shown potential in treating chronic pancreatitis, but may negatively impact diabetes management by reducing satiety signals, decreasing insulin secretion, and accelerating gastric emptying. These adverse effects complicate the use of metformin and proglumide as standalone treatments for patients suffering from these conditions which are frequently co-morbid or become co-morbid.

The described methods and compositions address these challenges by combining proglumide and metformin, or their pharmaceutically acceptable salts, to treat pancreatic diseases, including type 3c diabetes and chronic pancreatitis. This combination leverages the beneficial effects of each drug while mitigating their individual drawbacks. By co-administering proglumide and metformin, the potentially negative effects of each drug on the other's target condition are counterbalanced, providing a more comprehensive and effective treatment strategy for patients with these frequently comorbid conditions.

As used herein, each of the following terms has the meaning associated with it as described below.

The articles “a” and “an” are used herein to refer to one or to more than one (i.e., at least one) of the grammatical object of the article. By way of example, “an element” means one element or more than one element.

As used herein, “plurality” means at least two.

As used herein, “FDA” means the United States Food and Drug Administration.

Any ranges cited herein are inclusive, e.g., “between 100 mg and 2,400 mg” includes compositions of 100 mg and 2,400 mg.

As used herein, a subject is “treated”, or subjected to “treatment”, when an earnest attempt is made to alleviate a medical disorder or disease. For example, a subject can be treated for a disorder by being administered a pharmacologic agent that is intended to alleviate the disorder, irrespective of whether the treatment actually is successful in alleviating the disorder.

As used herein, a disease or disorder or medical affliction is “alleviated” if either the severity of a symptom of the disease or disorder or medical affliction, the frequency with which such a symptom is experienced by a subject, or both, are reduced.

A “subject” of diagnosis or treatment is a human.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

As used herein, pain means “an unpleasant sensory and emotional experience associated with actual or potential tissue damage or described in terms of such damage”, which is the definition that has been given by the International Association for the Study of Pain.

Administering an active pharmaceutical ingredient such as proglumide or metformin on a “pulsed basis” is defined as a course of administration wherein over a period of 28 days, the active pharmaceutical ingredient is administered to a human subject on 16 or fewer days, and wherein there are at least two 4-day periods during said 28 days on which the active pharmaceutical ingredient is not administered to the subject at all over four successive days. The primary benefit of administering proglumide on a pulsed basis is to reduce compensatory responses that can produce habituation.

There are many forms of pancreatic disease, also referred to as pancreatic disorders, including but not limited to: type 3c diabetes, chronic pancreatitis, acute pancreatitis, pancreatic cancer, and exocrine pancreatic insufficiency. One pancreatic disease can often lead to a secondary pancreatic disease, and comorbidity is common.

Pancreatitis is inflammation of the pancreas. Pancreatitis can occur as acute pancreatitis, which typically has a rapid onset and often lasts only for days. Some people develop chronic pancreatitis, which is a long-lasting inflammation of the pancreas which can cause permanent damage to the pancreas, often causes scarring of pancreatic tissue, and may cause the pancreas to stop making digestive enzymes and insulin in very severe cases.

Type 3c diabetes is a form of secondary diabetes also known as pancreatogenic diabetes. Type 3c diabetes is caused by damage to the pancreas, typically from pancreatitis, pancreatectomy/trauma, neoplasia, cystic fibrosis, hemochromatosis, or fibrocalculous pancreatopathy. Type 3c diabetes has been estimated to account for at least 5% of diabetes cases around the world, and the majority of type 3c diabetes cases are caused by chronic pancreatitis. A significant fraction of people with chronic pancreatitis, perhaps around 50%, eventually develop type 3c diabetes. In other words, it is likely that any individual with type 3c diabetes has had chronic pancreatitis, and there is a reasonable chance that anyone with chronic pancreatitis will eventually develop type 3c diabetes.

While type 3c diabetes and chronic pancreatitis are distinct diseases with distinct pathologies, symptoms, and recommended treatments, they have substantial comorbidity. Unfortunately, the most common treatment for type 3c diabetes (i.e., metformin) can cause or exacerbate chronic pancreatitis, while a promising clinical treatment for chronic pancreatitis (i.e., proglumide) might be counterproductive in patients with diabetes. Given the extensive comorbidity of these two diseases, administration of a therapeutic agent that treats one condition but induces or exacerbates the other condition is not optimal. By co-administering proglumide and metformin, the potentially negative effects of metformin on chronic pancreatitis are swamped by the more positive effects of proglumide on chronic pancreatitis, and the potentially negative effects of proglumide on diabetes are swamped by the more positive effects of metformin on diabetes. Consequently, combination of metformin and proglumide, including a fixed dose combination, can reduce the risk associated with treatment of type 3c diabetes, pancreatitis, or a combination thereof, without significantly attenuating the beneficial effects of these pharmaceutical agents.

Proglumide is a pharmaceutical agent previously approved outside of the United States for treatment of gastric ulcers and marketed under several different names. Proglumide has been suggested for use to treat many additional medical conditions, including but not limited to chronic pancreatitis, pancreatic cancer, colon cancer, nonalcoholic steatohepatitis, and potentiating opioids to treat pain. Many clinical trials using proglumide have been conducted. However, proglumide has never been approved by the FDA for any condition.

Metformin, typically administered as metformin hydrochloride, IUPAC name 3-(diaminomethylidene)-1,1-dimethylguanidine hydrochloride, is one of the most widely used pharmaceutical agents. It has well-established benefits for treatment of type 2 diabetes. It is also being studied for use to extend lifespans in humans and is frequently used off-label.

CCK is a peptide hormone which has many functions, including stimulating the digestion of fat and protein, and is also an important neuropeptide in the central nervous system. Elevated CCK levels induce anxiety. There are two main types of CCK receptors found in the body, known as CCK-A receptors and CCK-B receptors, or alternatively as CCK1 receptors and CCK2 receptors. CCK-A receptors predominate in the gastrointestinal tract, while CCK-B receptors predominate in the central nervous system.

Exogenously administered CCK or its related decapeptide analogue cerulein have been used for many years to induce acute pancreatitis in animal models. CCK antagonists have been long suggested as being potentially useful for reversing acute pancreatitis.

CCK antagonists have shown potential in humans for treating pancreatitis, but have not been unambiguously demonstrated to be effective for pancreatitis. Proglumide has advantages over other CCK antagonists. It has been proposed as a potential treatment for chronic pancreatitis for decades, with some success in human patients with chronic pancreatitis. It has a long record of safety when used in humans. It is not particularly selective for one type of CCK receptor (i.e., CCK-A receptors or CCK-B receptors). It also is not a particularly potent CCK receptor antagonist, which can be advantageous given homeostatic regulation.

In one embodiment, a method is described for treating chronic pancreatitis in a human subject which comprises administering to the subject a therapeutically effective amount of proglumide and metformin, or pharmaceutically acceptable salts of either active pharmaceutical agent, wherein proglumide, or pharmaceutically acceptable salt thereof, is administered at a cumulative daily dosage of between 250 mg and 2,400 mg per day, and wherein metformin, or pharmaceutically acceptable salt thereof, is administered at a cumulative daily dosage of between 500 mg and 2,500 mg per day. Relative to treatment with proglumide alone, the combination of proglumide and metformin provides the important benefits of helping maintain normal blood sugar levels and reducing the likelihood of developing diabetes.

In one embodiment, a method is described for treating type 3c diabetes in a human subject which comprises administering to the subject a therapeutically effective amount of proglumide and metformin, or pharmaceutically acceptable salts thereof, wherein proglumide, or pharmaceutically acceptable salt thereof, is administered at a cumulative daily dosage of between 250 mg and 2,400 mg per day, and metformin, or pharmaceutically acceptable salt thereof, is administered at a cumulative daily dosage of between 500 mg and 2,500 mg per day. Relative to treatment with metformin alone, the combination of proglumide and metformin, when administered to humans with pancreatogenic diabetes, can provide important benefits including but not limited to maintaining or reversing impaired pancreatic function, preventing exacerbation of known or undiagnosed pancreatic disease, and reducing the risk of pancreatic cancer, which frequently develops in people with type 3c diabetes.

In one embodiment, a method is described for treating a human subject with both chronic pancreatitis and type 3c diabetes which comprises administering to the subject a therapeutically effective amount of proglumide and metformin, or pharmaceutically acceptable salt thereof, wherein proglumide, or pharmaceutically acceptable salt thereof, is administered at a cumulative daily dosage of between 250 mg and 2,400 mg per day, and metformin, or pharmaceutically acceptable salt thereof, is administered at a cumulative daily dosage of between 500 mg and 2,500 mg per day. Relative to treatment with proglumide alone, the combination of proglumide and metformin can provide important benefits including but not limited to helping maintain normal blood sugar levels, reducing diabetic complications, reducing hemoglobin A1C levels, lower body weight, and increased lifespans. Relative to treatment with metformin alone, the combination of proglumide and metformin can provide benefits including but not limited to: improved therapy for chronic pancreatitis, reduced pancreatic inflammation, reduced pancreatic fibrosis, reduced pain, and reduced risk of pancreatic cancer.

In one embodiment, a method is described for treating type 3c diabetes and chronic pancreatitis in a human subject by administering to the subject a fixed dose combination of 500 mg proglumide and 1,000 mg metformin hydrochloride on a twice-daily basis. This treatment can be administered on a pulsed basis; for example, administering the pharmaceutical combination on one out of every four days, or daily on a week-on, week-off basis. Alternatively, this treatment can be administered on a daily basis for an extended period of time such as a week, a month, a year, or for the lifetime of a patient. Administering metformin and proglumide on a pulsed basis can reduce the likelihood of habituation.

In one embodiment, a method is described for treating type 3c diabetes and chronic pancreatitis in a human subject by administering to the subject a fixed dose combination of 500 mg proglumide and 1,000 mg metformin hydrochloride on a once-daily basis. In another embodiment, the fixed dose combination is 600 mg proglumide and 600 mg metformin hydrochloride.

In another embodiment, a fixed dose combination in an extended-release formulation is administered once-daily to a subject with type 3c diabetes, wherein the fixed dose combination includes 1,000 mg proglumide and 600 mg metformin hydrochloride.

In one embodiment, a method is described for preventing pain or reducing pain associated with chronic pancreatitis. A human subject is administered a therapeutically effective amount of proglumide and metformin, or pharmaceutically acceptable salts thereof.

In one embodiment, a planned placebo-controlled clinical study includes four arms: placebo, proglumide alone (subjects administered 2×800 mg proglumide daily), metformin alone (subjects administered 2×600 mg metformin hydrochloride daily), and metformin plus proglumide (subjects administered 800 mg proglumide and 600 mg metformin hydrochloride twice daily). All clinical trial subjects will have diagnoses of both chronic pancreatitis and type 3c diabetes.

Primary outcomes for type 3c diabetes include glycemic control indicators such as HbA1c levels, fasting blood glucose levels, post-prandial blood glucose levels, incidence of hypoglycemia, and insulin levels. Secondary outcomes for type 3c diabetes include (i) measurement of digestive enzyme levels to assess pancreatic exocrine insufficiency and (ii) body weight. Primary outcomes to assess chronic pancreatitis in the subjects include pain severity (assessed using visual analog scales or numerical rating scales), frequency and duration of pain episodes, and exocrine function as measured by fecal elastase or direct pancreatic function tests to assess digestive enzyme production. Secondary outcomes to assess chronic pancreatitis include quality of life (measured by standardized questionnaires to assess overall health, mental well-being, and daily functioning) and pancreatic imaging using MRI, CT scans, or ultrasound to monitor changes in the structure of the pancreas, such as calcifications, ductal changes, or the presence of pseudocysts.

### Formulation of Pharmaceutical Compositions

Pharmaceutical compositions contemplated by the methods and compositions of the invention may be formulated and administered to a subject for treatment of the diseases or afflictions disclosed herein as described below.

The invention encompasses the preparation and use of pharmaceutical compositions comprising proglumide and metformin, or pharmaceutically acceptable salts thereof, as active ingredients useful for treating type 3c diabetes and/or chronic pancreatitis. Such pharmaceutical composition may consist of the active ingredient(s) alone, in a form suitable for administration to a subject, or the pharmaceutical composition may comprise the active ingredient and one or more pharmaceutically acceptable carriers, one or more additional active ingredients, or some combination of these. The active ingredient may be present in the pharmaceutical composition in the form of a physiologically acceptable ester or salt, such as in combination with a physiologically acceptable cation or anion, as is well known in the art.

The compounds of the invention are also useful when formulated as salts. In cases where compounds are sufficiently basic or acidic to form stable non-toxic acid or base salts, administration of the compounds as salts may be appropriate. Examples of pharmaceutically acceptable salts are organic acid addition salts formed with acids which form a physiological acceptable anion, for example, tosylate, methanesulfonate, acetate, citrate, malonate, tartrate, succinate, benzoate, ascorbate, α-ketoglutarate, and α-glycerophosphate. Suitable acid addition salts of inorganic acids may also be formed, including hydrochloride, sulfate, nitrate, bicarbonate, and carbonate salts. Pharmaceutically acceptable salts may be obtained using standard procedures well known in the art, for example, by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

As used herein, the term “pharmaceutically acceptable carrier” means a chemical composition with which the active ingredient may be combined and which, following the combination, can be used to administer the active ingredient to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

The descriptions of pharmaceutical compositions provided herein are directed to pharmaceutical compositions which are suitable for ethical administration to humans.

Pharmaceutical compositions that are useful in the methods of the invention may be prepared, packaged, or sold in formulations suitable for oral, rectal, vaginal, parenteral, sublingual, topical, pulmonary, intranasal, buccal, ophthalmic, intrathecal or another route of administration. Other contemplated formulations include projected nanoparticles, liposomal preparations, resealed erythrocytes containing the active ingredient, and immunologically based formulations.

A pharmaceutical composition of the invention may be prepared, packaged, or sold in bulk, as a single unit dose, or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

A pharmaceutical kit may be utilized for pulsed dosing which contains several weeks supply of formulated pills comprising the fixed dose combination, in addition to inactive pills to be taken on days when proglumide and metformin are not administered according to the methods of the invention.

The relative amounts of the active ingredients, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

The therapeutically effective dose of the pharmacologic agent can be administered using any medically acceptable mode of administration. Although the skilled artisan would contemplate any of the modes of administration known to one of ordinary skill, preferably the pharmacologic agent is administered according to the recommended mode of administration, for example, the mode of administration listed on the package insert of a commercially available agent.

Controlled-release, extended-release, or sustained-release formulations of a pharmaceutical composition of the invention may be made using conventional technology, including any methods known in the art useful for extending the release of active pharmaceutical ingredients from a pharmaceutical delivery system such as a tablet or capsule.

A formulation of a pharmaceutical composition of the invention suitable for oral administration may be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion.

As used herein, an “oily” liquid is one which comprises a carbon-containing liquid molecule and which exhibits a less polar character than water.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface-active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycolate. Known surface active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Tablets may be non-coated or they may be coated using known methods to achieve delayed disintegration in the gastrointestinal tract of a subject, thereby providing sustained release and absorption of the active ingredients. By way of example, a material such as glyceryl monostearate or glyceryl distearate may be used to coat tablets. Further by way of example, tablets may be coated using methods described in U.S. Pat. Nos. 4,256,108; 4,160,452; and U.S. Pat. No. 4,265,874 to form osmotically-controlled release tablets. Tablets may further comprise a sweetening agent, a flavoring agent, a coloring agent, a preservative, or some combination of these in order to provide pharmaceutically elegant and palatable preparation.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

Liquid formulations of a pharmaceutical composition of the invention which are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g. polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin and acacia. Known preservatives include, but are not limited to, methyl, ethyl, or n-propyl para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of a dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil in water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

A pharmaceutical composition of the invention may be prepared, packaged, or sold in a formulation suitable for rectal administration. Such a composition may be in the form of, for example, a suppository, a retention enema preparation, and a solution for rectal or colonic irrigation.

Suppository formulations may be made by combining the active ingredient with a non-irritating pharmaceutically acceptable excipient which is solid at ordinary room temperature (i.e., about 20° C.) and which is liquid at the rectal temperature of the subject (i.e., about 37° C. in a healthy human). Suitable pharmaceutically acceptable excipients include, but are not limited to, cocoa butter, polyethylene glycols, and various glycerides. Suppository formulations may further comprise various additional ingredients including, but not limited to, antioxidants and preservatives.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying.

As used herein, “parenteral administration” of a pharmaceutical composition includes any route of administration characterized by physical breaching of a tissue of a subject and administration of the pharmaceutical composition through the breach in the tissue, including intramuscular, subcutaneous, intravenous, and intradermal injection. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, intravenous, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally acceptable diluent or solvent, such as water or 1,3-butanediol, for example. Other acceptable diluents and solvents include, but are not limited to, Ringer's solution, isotonic sodium chloride solution, and fixed oils such as synthetic mono- or di-glycerides. Other parenterally administrable formulations which are useful include those which comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

Formulations suitable for topical administration include, but are not limited to, liquid or semi liquid preparations such as liniments, lotions, oil in water or water in oil emulsions such as creams, ointments or pastes, and solutions or suspensions. Topically administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally, the propellant may constitute 50% to 99.9% (w/w) of the composition, and the active ingredient may constitute 0.1% to 20% (w/w) of the composition. The propellant may further comprise additional ingredients such as a liquid non-ionic or solid anionic surfactant or a solid diluent (preferably having a particle size of the same order as particles comprising the active ingredient).

A pharmaceutical composition of the invention may be prepared, packaged, or sold in a formulation suitable for buccal administration. Such formulations may, for example, be in the form of tablets or lozenges made using conventional methods, and may, for example, 0.1% to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 to about 200 nanometers and may further comprise one or more of the additional ingredients described herein.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed., 1985, Remington's Pharmaceutical Sciences, Mack Publishing Co., Easton, PA, which is incorporated herein by reference.

### Dosage

For treating pancreatic disease, proglumide (or salt thereof) dosage levels can be between about 100 mg and 2,400 mg when administered to adult subjects, while metformin (or salt thereof) dosage levels can be between about 250 mg and 2,500 mg. The therapeutically effective doses of proglumide and metformin can be administered using any medically acceptable mode of administration. Although the skilled artisan would contemplate any of the modes of administration known to one of ordinary skill, preferably the pharmacologic agent is administered according to the recommended mode of administration, for example, the mode of administration listed on the package insert of a commercially available agent.

In one embodiment, fixed dose combination tablets useful for treating pancreatic disease, including type 3c diabetes and/or chronic pancreatitis can be made by: (1) mixing 1,200 g proglumide, 1,300 g metformin hydrochloride, 600 g lactose, and 700 g starch, then adding 300 g of wet starch and mixing to homogeneity; (2) grounding the resultant mass while wet, passing through a 15 mesh sieve, and mixing with 50 g talc, 20 g magnesium stearate, and 30 g silicon dioxide; and (3) compressing the mixture in a rotary compressor and tableting to provide 10,000 tablets each weighing 420 mg and comprising 120 mg proglumide, 130 mg metformin hydrochloride, 60 mg lactose, 100 mg starch, 5 mg talc, 2 mg magnesium stearate, and 3 mg silicon dioxide. Smaller or larger tablets can be made from the same mixture.

In another embodiment, a mixture of 45 g of proglumide, 30 g of metformin hydrochloride, 75 g of lactose and 100 g of talc can be combined, mixed, wetted with a sufficient quantity of alcohol and granulated followed by drying. The obtained granulate can be filled into capsules containing, for example, 400 mg metformin hydrochloride and 600 mg proglumide. The pharmaceutical compositions can be useful for treating human subjects with (i) chronic pancreatitis, (ii) type 3c diabetes, or (iii) chronic pancreatitis and type 3c diabetes.

### Timing and Frequency of Dosage

According to the methods described, proglumide and metformin (or pharmaceutically acceptable salts thereof) can be administered to a patient on a once-daily basis, a twice-daily basis, or a thrice-daily basis for any period.

If pulsed administration is used, then in any 28-day period, metformin and proglumide should be administered on no more than 16 days, and there should be at least two periods of four or more consecutive days wherein proglumide is not administered.

For example, in one exemplary embodiment of pulsed administration, to a human subject with both type 3c diabetes and chronic pancreatitis, a fixed dose combination of proglumide and metformin hydrochloride is administered for a week at a time, separated by periods of seven or more days when the fixed dose combination is not administered.

### INCORPORATION BY REFERENCE

All publications, patents, and patent applications cited herein are hereby expressly incorporated by reference in their entirety and for all purposes to the same extent as if each was so individually denoted.

### EQUIVALENTS

While specific embodiments of the subject invention have been discussed, the above specification is illustrative and not restrictive. Many variations of the invention will become apparent to those skilled in the art upon review of this specification. The full scope of the invention should be determined by reference to the claims, along with their full scope of equivalents, and the specification, along with such variations.

1. A method for treating pancreatic disease in a human subject which comprises administering to the subject (i) a therapeutically effective amount of proglumide, or pharmaceutically acceptable salt thereof, and (ii) a therapeutically effective amount of metformin, or pharmaceutically acceptable salt thereof;
wherein said proglumide, or pharmaceutically acceptable salt thereof, is administered at a dosage of between 250 mg and 2,400 mg per day; and
wherein said metformin, or pharmaceutically acceptable salt thereof, is administered at a dosage of between 500 mg and 2,500 mg per day.

2. The method of claim 1, wherein said method is used for the treatment of chronic pancreatitis.

3. The method of claim 1, wherein said method is used for the treatment of type 3c diabetes.

4. The method of claim 1, wherein said human subject has both type 3c diabetes and chronic pancreatitis.

5. The method of claim 1, wherein said human subject has post-pancreatis diabetes mellitus.

6. The method of claim 1, wherein at least one of said proglumide, or pharmaceutically acceptable salt thereof, and said metformin, or pharmaceutically acceptable salt thereof, is administered as an extended-release formulation.

7. The method of claim 1, wherein said proglumide, or pharmaceutically acceptable salt thereof, and said metformin, or pharmaceutically acceptable salt thereof, are administered on a pulsed basis.

8. The method of claim 1, wherein said subject receives a therapeutic benefit selected from the group consisting of: reduced risk of developing diabetes, reduced risk of developing cancer, reduced risk of exacerbating pancreatitis, improved management of blood sugar levels, reduced hemoglobin A1C percentage, reduced average blood sugar levels, reduced post-prandial blood sugar levels, fewer hyperglycemic episodes, fewer hypoglycemic episodes, reduced pain, reduced pancreatic inflammation, reduced pancreatic fibrosis, and reduced risk of developing pancreatic cancer.

9. A pharmaceutical composition useful for treating a pancreatic disease comprising proglumide, or pharmaceutically acceptable salt thereof, and metformin, or pharmaceutically acceptable salt thereof.

10. The pharmaceutical composition of claim 9, wherein the dosage of proglumide, or pharmaceutically acceptable salt thereof, is between 100 mg and 2,400 mg, and wherein the dosage of metformin, or pharmaceutically acceptable salt thereof, is between 250 mg and 2,500 mg.

11. The pharmaceutical composition of claim 9, wherein the pancreatic disease is selected from the group consisting of type 3c diabetes and chronic pancreatitis.

12. The pharmaceutical composition of claim 9, wherein the pancreatic disease is type 3c diabetes.
